(19) United States
(12) Patent Application Publication
(10) Pub. No.: US 2016/0101093 A1
(43) Pub. Date: Apr. 14, 2016

(54) METHODS FOR THE TREATMENT OF PREMATURE EJACULATION

(71) Applicant: Jason B. WYNBERG, Southfield, MI (US)

(72) Inventor: Jason B. WYNBERG, Oak Park, MI (US)

(21) Appl. No.: 14/893,019

(22) PCT Filed: May 21, 2014
(86) PCT No.: PCT/US2014/038985

§ 371 Date: Nov. 20, 2015

Related U.S. Application Data
(60) Provisional application No. 61/989,678, filed on May 7, 2014.
(60) Provisional application No. 61/903,712, filed on Nov. 13, 2013.
(60) Provisional application No. 61/902,860, filed on Nov. 12, 2013.
(60) Provisional application No. 61/897,316, filed on Oct. 30, 2013.
(60) Provisional application No. 61/897,070, filed on Oct. 29, 2013.
(60) Provisional application No. 61/829,099, filed on May 30, 2013.
(60) Provisional application No. 61/825,776, filed on May 21, 2013.

(51) Int. Cl. A61K 31/445; A61M 37/00; A61M 5/32; A61M 5/30; A61K 9/00; A61K 31/135

(52) U.S. Cl. CPC A61K 31/445; A61K 9/0019; A61K 9/0034; A61K 31/135; A61M 5/329; A61M 5/30; A61M 37/0015

(57) ABSTRACT

The invention relates to methods of treating premature ejaculation in a patient in need thereof, wherein the methods comprise administering an effective amount of a solution comprising a local anesthetic and/or SSRI, and wherein the solution is injected directly into the penis of the patient. The invention is also related to kits comprising an injection system for physician office/home and/or personal use.

### CROSS REFERENCE TO RELATED APPLICATIONS

This application claims priority to prior filed U.S. provisional application 61/825,776, filed on May 21, 2013, and U.S. provisional application 61/829099, filed May 30, 2013, and U.S. provisional application 61/897,070, filed Oct. 29, 2013, and U.S. provisional application 61/897,316, filed Oct. 30, 2013 and U.S. provisional application 61/902,860, filed Nov. 12, 2013, and U.S. provisional application 61/903,712, filed Nov. 13, 2013, and U.S. provisional application 61/989,678, filed May 7, 2014, the contents of each of which are incorporated herein by reference in their entireties.

### FIELD OF THE INVENTION

The field relates to methods of treating premature ejaculation, wherein the method comprises administration of a local anesthetic, SSRI, and/or opioid to a patient in need thereof. The field further relates to methods of treating premature ejaculation, wherein a local anesthetic and/or SSRI is administered by an injection, e.g, needle injection, needle-free injection (e.g., needle free jet injection), or microneedle injection.

### BACKGROUND

Premature ejaculation (PE) is a common sexual dysfunction in men. Premature ejaculation can be generally defined as the occurrence of ejaculation prior to or sooner than hoped for by one or both sexual partners. As one example, premature ejaculation can be experienced as ejaculation before, upon or shortly after penile penetration of a sexual partner. If the instances of premature ejaculation are few and far between, then such occurrences may not be a cause for concern. However, if instances of premature ejaculation occur practically every time intercourse is attempted, or even if premature ejaculation occurs even greater than about 10% or about 20% of the time intercourse is attempted, then treatment of the condition is likely to be warranted.

The prevalence rate of premature ejaculation in American males is estimated to range from 5-30%. Premature ejaculation is often a lifelong condition for sexually active men. It can otherwise occur at virtually any age in an adult man's life. It is most commonly reported by younger men (aged 18-30 years old) and is also commonly reported in conjunction with secondary impotence in men aged 45-65 years.

Various compositions can be utilized for treating premature ejaculation. In some cases selective serotonin reuptake inhibitors (SSRI's) may be used for treating premature ejaculation. Additional methods for treating premature ejaculation may include administration of an effective amount of a tramadol (an opioid analgesic) material to a male prior to sexual intercourse, as well as delaying the onset of ejaculation in a male by systemically administering to the individual a rapid-release pharmaceutical formulation containing clomipramine and pharmacologically acceptable acid addition salts thereof. Other techniques include: clomipramine (tricyclic antidepressant) prescribed oral treatment with SSRI (selective serotonin reuptake inhibitor) e.g. sertraline, fluoxetine or paroxetine (taken daily or ‘as needed’) and the application of topical anesthetics, such as lidocaine 2% cream, for example, to the penis before intercourse.

There are significant drawbacks to many of these applications and methods. For instance, the oral administration of SSRI's essentially constitute a systemic treatment of a local issue, can cause fatigue, and do not typically act immediately. Treatment with topical creams and ointments can take a significant amount of time to work. Additionally, their residue can create an unwanted tingling sensation in the partner and can leave an unwanted taste, negatively affecting oral sexual activity and making it more difficult to conceal the use of the cream from the partner. Accordingly, there is a need for a treatment for premature ejaculation that is: efficacious, fast-acting, can be used locally, e.g., directly applied to the penis, doesn't cause sensations of penile numbness or taste, and is not present when it is not needed i.e. between sexual experiences. Moreover, it would be important for such a treatment to be administered by a patient and used in the privacy and comfort of their home without having to regularly see a physician for administration of a particular drug or compound.

### BRIEF DESCRIPTION OF THE FIGURES

FIG. 1a is a top view of the dorsum of the shaft of the penis. In one aspect the injection can take place either to the left or right of the midline.

FIG. 1b is a side view of the penis. In one aspect the injection can take place in the area of the frenulum and/or in the area of the dorsum.

FIG. 1c is a side view of the penis. In one aspect the injection can take place near the abdominal wall and, in one aspect is an infrapubic injection.

### SUMMARY OF THE INVENTION

Despite the long-felt need for an efficacious and fast-acting way to treat premature ejaculation there is nothing that sufficiently addresses this issue. Typically, standard treatment may entail the prescription and administration of an anti-anxiety pill or possibly the administration of a topical anesthetic.

One hypothesis for the cause or pathobiology of premature ejaculation is that of hypersensitivity of sexual stimulation. Hypersensitivity of sexual stimulation is hypothesized to be caused by a disorder in the complex cooperation between the peripheral nervous system and the central nervous system. Hypersensitivity of sexual stimulation is believed to lead to the onset of ejaculation before or shortly after vaginal penetration, or an inability to keep erection or control ejaculation for a sufficient amount of time for a partner's sexual pleasure.

Without being bound by any theory, the present invention is believed to work by reducing signal conduction along the dorsal nerves of the penis by anesthetizing these nerves. This type of anesthesia may serve to reduce sensation of the glans penis and part of the penis (depending on how distal on the penis the injection is placed). In certain aspects, the invention contemplates avoiding vessels e.g. midline superficial and deep dorsal veins, and arteries. Furthermore, in certain aspects alternate therapeutic targets may additionally include branches of the perineal nerve serving the glans penis and frenulum.

In certain aspects the depth of the injection is into the subcutaneous space superficial to Buck's fascia. Without being bound by any theory, it is believed that diffusion of therapeutic drug (e.g. local anesthetic) to the sensory dorsal penile and/or perineal nerves and/or branches of these nerves is sufficient to lessen the hypersensitivity which may be the cause of the premature ejaculation. In another aspect, the invention contemplates injections within the Buck's fascia in order to target the nerves directly. In certain aspects, the injection within the Buck's fascia may be performed under ultrasound guidance. The invention contemplates that the depth of injection can be controlled by selecting which injection system is employed, power of the injection (for needle-free devices), and length of needle (for needle-based devices). Some inject to dermis, some to subcutaneous fat, and some deep to Buck's fascia. In one aspect, the invention contemplates that the depth of injection is the subcutaneous space.

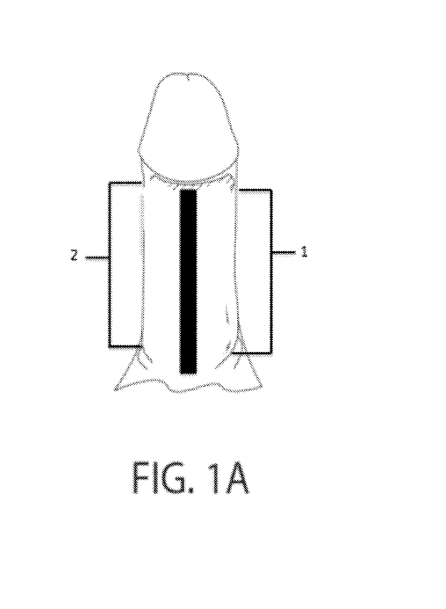

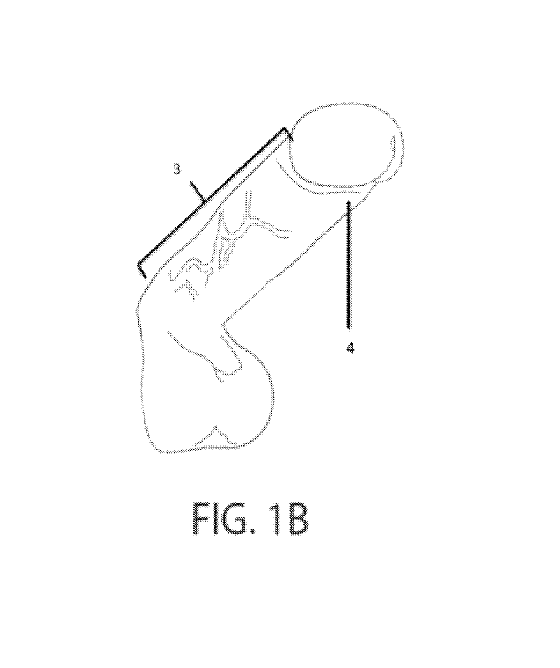

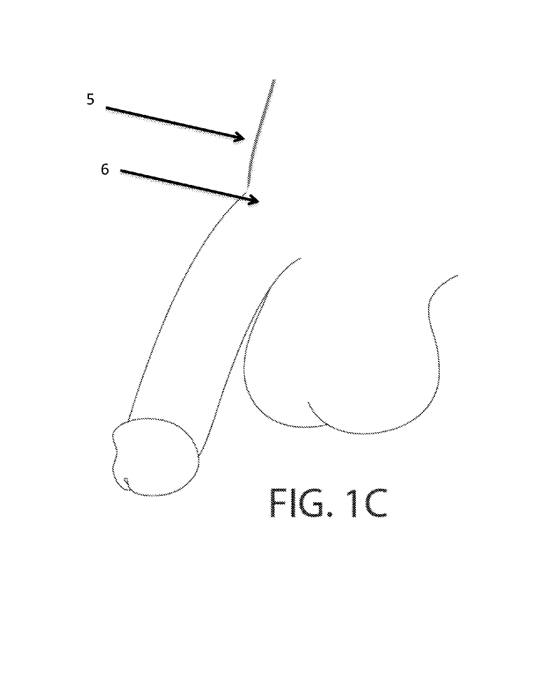

It is an advantage of the present invention that the disclosed methods (e.g., any of Method 1.0 et seq.) delivers a predetermined and therapeutically effective amount of active substances (e.g., solution comprising local anesthetics and/or SSRI) to temporarily reduce conduction of the sensory nerves of the glans and/or penis to cause desensitization of these nerves, interruption of the neuronal pathways associated with and underlying premature ejaculation, and/or interruption of muscles involved in the orgasm/ejaculation pathway (e.g. bulbospongiosus muscle). Other target sites may include glans penis, frenulum, and bulbospongiosus muscles. In one aspect, the invention advantageously reduces sensation or stimulation without a total penile block, thus retaining penile sensation for sexual pleasure while reducing sensation enough to extend time to ejaculation. In yet another aspect, the invention contemplates a subcutaneous injection to the penis. In this aspect it is believed that the injected solution (e.g., local anesthetic and/or SSRI and/or opioid) transiently treats/anesthetizes the dorsal penile nerves near the dorsal midline of penis, and/or the branches of the dorsal penile nerves just before the glans penis along the broader dorsal surface of the penis. Thus, in one aspect the location of the injection is on the dorsum of the penis, e.g., between the base of the penis to the coronal sulcus (See, FIG. 1a). In another aspect, the site of injection includes the ventrum of the penis, frenulum (See, FIG. 1b) or infrapubic injection (See, FIG. 1c).

Thus, the present invention addresses the need for a fast acting, efficacious, self-limited in time, and private way to treat male premature ejaculation. In one aspect the present invention allows a patient to inject themselves in the privacy of their home or without the assistance of a medical professional.

In one aspect the present invention provides for METHOD 1.0, wherein the method is directed toward a method of treating premature ejaculation in a patient in need thereof, wherein the method comprises administering a an effective amount of a solution comprising a local anesthetic, opioid, and/or SSRI, wherein the solution is injected directly into the penis.

For example, Method 1.0 encompasses the following:

1.1 Method 1.0, wherein the local anesthetic, opioid, and/or SSRI is administered between about 30 seconds to 12 hours (and up to 24 hours) prior to engaging in sexual intercourse, e.g., 1, 2, 3, 4, 5, 6, 7, 8, 9, 10, 20, 30 minutes prior to sexual intercourse, e.g., 1, 2, 3, 4, 5, 6, 7, 8, 9, 10, 11, 12 hours and up to 24 hours prior to sexual intercourse.

1.2 Method 1.0 or 1.1, wherein the patient self-administers the anesthetic and/or SSRI prior to engaging in sexual activity.

1.3 Method 1.0, 1.1, or 1.2, wherein the local anesthetic is selected from the group consisting of: Lidocaine, marcaine, tetracaine, bupivacaine, mepivacaine, Articaine, Carticaine, Cinchocaine/Dibucaine, chlorprocaine, dibucaine, etidocaine, hexylcaine, procaine, ketamine, pramoxine, dyclonine and phenol, Levobupivacaine, Piperocaine, Ropivacaine, Trimecaine, Benzocaine, Chloroprocaine, Cocaine, Cyclomethycaine, Dimethocaine/Larocaine, carbisocaine, ciprocaine, butanilicaine and trimecaine, Propoxycaine, Procaine, Prilocaine, Proparacaine, Tetracaine/Amethocaine, Saxitoxin and Tetrodotoxina.

1.4 Any of the preceding methods, wherein the local anesthetic is lidocaine.

1.5 Any of the preceding methods, wherein the local anesthetic is bupivacaine.

1.6 Any of the preceding methods, wherein the SSRI is selected from the group consisting of: Paroxetine, dapoxetine, Clomipramine, Fluoxetine, sertraline, flibanserine.

1.7 Any of the preceding methods wherein the injection further comprises opioids e.g. Tramodol.

1.8 Any of the preceding methods wherein the injection is administered using a needle injection directly to the penis.

1.9 The method of 1.8, wherein the gauge of the needle is greater than 23, e.g., 24, 25, 26, 27, 28, 29, 30, 31, 32, 33, 34, 35, 36, or 37 gauge.

1.10 The method of 1.8 or 1.9 wherein the length of the needle is between about 3-10 mm, e.g., 3 mm, 4 mm, 5 mm, 6 mm, 7 mm, 8 mm, 9 mm or 10 mm.

1.11 The method of any of 1.8-1.10, wherein the gauge of needle is between 30-32, and the length of the needle is between 4-7 mm.

1.12 Any of the preceding methods, wherein the injection is administered using a pre-filled disposable single use syringe, (e.g., SIMPLIST™ from Becton Dickenson)

1.13 The method of method 1.12, wherein the injection is administered via a pre-filled syringe (e.g., glass (e.g., Becton Dickson Hypak™; plastic (e.g, Becton Dickson Sterifill™; Becton Dickson Vystra™ Disposable pen; Becton Dickson Pen II Reusuable pen; Becton Dickson Physiojet™ Disposable Autoinjector; cyclo olefin polymer (COP); cyclo olefin copolymer (COC) (e.g., Topas))

1.14 Any of the preceding methods, wherein the injection is administered using an auto-injection pen (e.g., Apidra SOLOSTAR™, Lantus SOLOSTAR™, BYETTA™, SYMLINPEN 60™, HumaPen LUXURA HD™, Humalog KWIKPEN™, Humulin Pen, VICTOZA™, NOVOLOG MIX FLEXPEN™, NOVOPEN JUNIOR™, NOVO PEN 3™, LEVEMIR FLEXPEN™, AUTOPEN™, Follistim pen, and CAVERJECT IMPULSE™.

1.15 Any of the preceding methods, wherein the injection is administered via a microneedle injection, e.g., a microneedle dermal patch, e.g., 3M HOLLOW MICRONEEDLE SYSTEM™ or 3M SOLID MICRONEEDLE SYSTEM™.

1.16 Any of the preceding methods, wherein the injection is administered via a needle-free jet injection (e.g., using a BIOJECT™ needle-free jet injector or Pharmajet, for example).

1.17 Any of the preceding methods, wherein the volume of the solution is between about 0.05 ml-2.0 ml, e.g., about 0.05 ml, 0.1 ml, 0.2 ml, 0.3 ml, 0.4 ml, 0.5 ml, 0.8 ml, 1.0 ml, 1.5 ml, or 2.0 ml.

1.18 Any of the preceding methods, wherein the injection is administered via transdermal, intramuscular, subcutaneous, subdermal, intradermal or implant.

1.19 Any of the preceding methods, wherein the injection is a subcutaneous injection.

1.20 Any of the preceding methods, wherein the injection is administered in combination with an EMLA (“Eutectic Mixture of Local Anesthetics”) gel.

1.21 Any of the preceding methods, wherein the volume of the therapeutically effective amount is between about 0.1 mL to 1.0 mL, e.g., 0.1 mL, 0.2 mL, 0.3 mL, 0.4 mL, 0.5 ml, 0.6 ml, 0.7 ml, 0.8 ml, 0.9 ml, 1.0 ml.

1.22 Any of the preceding methods wherein the local anesthetic is 0.25%-1% Bupivacaine (marcaine) or 1-2% lidocaine.

1.23 Any of the preceding methods wherein the local anesthetic is 0.05%-1% Bupivicaine (marcaine) or 0.2% 1-22% lidocaine.

1.24 The method of any of 1.21-1.23, wherein the local anesthetic is 0.25% bupivacaine.

1.25 Any of the preceding methods wherein the local anesthetic and/or SSRI is administered in combination with Botox, and wherein the administration takes place immediately prior to sexual activity.

1.26 Any of the preceding methods wherein a test dose is administered is administered prior to longer duration therapies, e.g., botox, surgical denervation, neuromodulation, cryoablation, radiofrequency ablation, and wherein the purpose of the test dose is to determine patient satisfaction and/or to optimize therapy plan for longer duration therapies.

1.27 The method of 1.26, wherein the test dose aides in optimizing the location of the therapy, extent, dose, and/or side effects of longer duration therapy.

1.28 Any of the preceding methods, wherein the needle gauge is 32 and the length is between 4 and 10 mm.

1.29 Any of the preceding methods, wherein the injection is administered subcutaneously to the dorsum of the penis anesthetizing the dorsal penile nerves near and/or around the dorsal midline of penis, or the branches of the dorsal penile nerves just before the glans penis along the broader dorsal surface of the penis.

1.30 Any of the preceding methods, wherein the injection is administered subcutaneously to the ventrum of the penis and/or frenulum.

1.31 Any of the preceding methods, wherein the injection is administered as an infrapubic injection.

1.32 Any of the preceding methods, wherein the injection system is an extended drug delivery system which provides prolonged therapy.

1.33 The method of 1.32 wherein the extended delivery system is in the form of an implant and/or injection (e.g., single injection)

1.34 The method of 1.33, wherein the extended delivery system in selected from the group consisting of: polymer systems, polymeric matrices, organogels, reservoir matrices, matrix diffusion-controlled devices, nanostructured lipid carriers (NLC), multilayer matrix assemblies, hydrogels (e.g., semi-solid assemblies, e.g., VANTAS™ system for LHRH (ENDOPHARM™, poly(vinyl alcohol)-tetraborate hydrogel systems, (e.g., lidocaine delivery), multivesicular liposomes (MVL) (e.g., lipid vesicules having multiple non-concentric internal aqueous chambers having internal membranes distributed as a network throughout the MVL, examples can be found in U.S. Pat. No. 8,182,835 the contents of which are incorporated herein by reference in their entirety.)

1.35 The method of 1.34, wherein a local anesthetic is administered via a liposome delivery system.

1.36 The method of 1.35, wherein bupivacaine is administered in a liposome delivery system.

1.37 The method of 1.33, wherein a local anesthetic is administered in a hydrogel delivery system.

1.38 The method of 1.37, wherein bupivacaine is administered in a hydrogel delivery system.

1.39 Any of the preceding methods, wherein the needle used is e.g. BD Micro-Fine™ Needles; BD Ultra-Fine™; Pen Needles; BD AutoShield™ Pen Needle.

1.40 Any of the preceding methods, wherein the injection is administered using a patch injector (e.g. Becton Dickson™ Microinfusor Patch Injector.

1.41 Any of the preceding methods, wherein the method utilizes a safety needle system (e.g., Beckton Dickson Preventis™; Becton Dickson SafetyGlide™; Becton Dickson Eclipse™)

1.42 Any of the preceding methods wherein the injection is selected from the group consting of: transdermal, intramuscular, subcutaneous, subdermal, intradermal, parenteral and implant.

1.43 The method of method 1.42, wherein the administration can be dorsally (e.g., frenulum or prepuce).

1.44 The method of any of the preceding methods, wherein the injection is used to administer medications relating to the treatment of impotence (e.g., prostaglandin).

1.45 The method of 1.44, wherein the medication related to the treatment of impotance is administered in combination with a local anesthetic (e.g., lidocaine, bupivacaine)

The invention also provides for a KIT 2.0, wherein the kit comprises an injection system (e.g., standard needle injection (e.g., needle injection), prefilled syringe, pen needle system, microneedle injection, or needle-free jet injection), 1) syringe or needle-free injector; (2) medication in liquid or powder (e.g., lyophilized) form; (3) diluent if medication in powder form; (4) if needle-based then thin, short needle for injection (e.g., 30 gauge 4-10 mm); (5) needle for drawing up medication if standard needle injection system; (6) alcohol swab for skin and to clean vials (as needed depending on kit composition) prior to drawing up medication; (7) instructions for use with diagrams. Wherein the kit offers the benefit of allowing a patient to administer the medication himself or herself.

For example Kit 2.0 encompasses the following:

2.1 Kit 2.0 wherein the syringe is a prefilled syringe.

2.2 Kit 2.1 where the prefilled syringe is a preloaded with a 32 gauge, 6 mm long needle.

2.3 Kit 2.0-2.2 wherein the syringe is prefilled with 1 mL of 0.25% bupivacaine.

2.4 Any of Kit 2.0-2.3 wherein the kit contains an alcohol swab.

2.5 Any of Kit 2.0-2.4 wherein the syringe is a prefilled syringe

2.6 Any of Kit 2.0-2.5 wherein the kit is used in combination with any of Method 1.0 et seq.

In one aspect the invention contemplates a kit (e.g., any of Kit 2.0 et seq) wherein the kit contains instructions for use. In one aspect, the instructions include steps in the following order: 1. Wipe dorsum of penis, or frenulum; 2. Uncap needle, inject, and dispose of needle. In one aspect, the Kit of 2.0 allows a patient to administer the medication in the privacy of their home and/or without the assistance of a medical professional.

### DETAILED DESCRIPTION

As used herein, the words or terms set forth below have the following definitions.

“Therapeutically effective amount,” or “effective amount” as used herein, means an amount of a local anesthetic, opioid, or SSRI, that ameliorates, or eliminates one or more symptoms of a particular disease or condition (e.g., premature ejaculation) or prevents or delays the onset of one or more symptoms of a particular disease or condition.

In accordance with this detailed description, the following abbreviations and definitions apply. It must be noted that as used herein, the singular forms “a”, “and”, and “the” include plural referents unless the context clearly dictates otherwise. Thus, for example, reference to “a compound” includes a plurality of such compounds and reference to “the dosage” includes reference to one or more dosages and equivalents thereof known to those skilled in the art, and so forth.

The term “subject” or “patient” as used herein is meant to include a mammal. In a preferred aspect of the present invention the mammal is a human.

The terms “disorder,” “disease,” and “condition” are used inclusively and refer to any status deviating from normal.

“Local administration” means direct administration by a non-systemic route at or in the vicinity of the site of an affliction or disorder (e.g., directly to the penis). Thus, as described in any of Methods 1.0 et seq., local administration of a pharmaceutical comprising a local anesthetic, opioid, or SSRI excludes intravenous or oral administration, but includes, for example, intramuscular, transdermal or subcutaneous injection or placement of a sustained-release implant for delivery of the local anesthetic or SSRI.

“Treating” means to alleviate (or to eliminate) at least one symptom, either temporarily or permanently. Here, this includes increasing the time (i.e. prolongation of climax time) it takes a patient to reach climax after sexual arousal. In a particular example, climax time is the time between the start of intercourse and the time at which climax is achieved.

“Climax baseline time” is the pre-treatment climax time of a patient, that is, the time or average time that it takes for a patient to climax after becoming sexually aroused.

“Prolongation of climax time” means an increase in time (increase in climax baseline time) from which a patient becomes sexually aroused to the time of sexual climax (i.e. orgasm). This is commonly measured as the ‘intravaginal latency time’ and is measured by stopwatch held and activated by the female partner commencing with insertion of male organ into the female, ending at climax of the male. In one aspect, “treating premature ejaculation” means increasing the time between the beginning of sexual arousal of a patient and ejaculation by the patient; and in particular instances, it can mean increasing the time from which sexual intercourse begins to the time of ejaculation.

“SSRI” as used herein refers to the selective serotonin re-uptake inhibitors or serotonin-specific reuptake inhibitor (SSRIs) class of compounds typically used as antidepressants in the treatment of depression, anxiety disorders, and some personality disorders. “SSRI's” may include, e.g., citalopram, dapoxetine, escitalopram, fluoxetine, fluvoxamine, indalpine, paroxetine, sertraline, zimelidine.

“Test dose” as used herein refers to the administration of a local anesthetic, opioid, and/or SSRI, prior to longer duration therapies, e.g., botox, surgical denervation, neuromodulation, cryoablation, radiofrequency ablation, wherein the purpose of the test dose is to determine, prior to committing to longer duration therapies, the (1) patient satisfaction with this intervention, (2) side-effect profile (e.g. numbness, extent of penile sensation loss, etc.), (3) optimize dose/extent of treatment and optimize location of treatment prior to committing to longer duration therapies. Such test doses may be one-time or multiple-occasion tests and may be performed at various sites on the penis to determine optimal site and/or extent of therapy for more durable intervention.

The terms “standard needle injection” and “needle injection” may be used interchangeably throughout the description.

“Microneedle” as used herein may refer to needles for injectable devices which are about 150 μm-950 μm in height. In one aspect, a microneedle is the HOLLOW MICRONEEDLE SYSTEM (hMTS)™ sold by 3M.

The solution comprising the SSRI and/or local anesthetic and/or opioid, as described in any of Method 1.0 et seq., with the goal to prolong intravaginal latency time to where subjectively one or both partners notice a definite prolongation of intravaginal latency time, and/or to where the true, measured intravaginal latency time is extended at least two-fold from baseline e.g. 45 seconds to 90 seconds. Ideally, this intervention would prolong intravaginal latency time to more than 3-4 minutes.

In another embodiment (e.g., any of Method 1.0 et seq.), a method for treating premature ejaculation in a patient in need thereof is provided, where the method comprises a step of locally administering, by injection, a local anesthetic, opioid, and/or SSRI to a penis of the patient, thereby treating premature ejaculation in the patient. In certain aspects, the local anesthetic or SSRI is injected into at least two penile locations, in some aspects at least three penile locations. In specific examples, local administration of local anesthetic or SSRI is believed to affect the dorsal penile nerves or branches thereof, the perineal nerve or branches thereof, injected into the dorsum of penis, frenulum or glans of the penis.

In any of the methods disclosed herein (e.g., any of Method 1.0 et seq), the solution comprising the SSRI, opioid, and/or local anesthetic may further comprise pharmaceutically acceptable acidifying agents and/or alkalizing agents and/or buffers for adjusting and stabilizing the pH of the solutions. Acidifying agents may include inorganic acids and/or organic acids and/or inorganic salts and/or organic salts.

Alkalizing agents may include inorganic bases and/or organic bases and/or inorganic salts and/or organic salts. Examples of acidifying agents may be but are not limited to hydrochloric acid, carbonic acid, phosphoric acid, histidine HCl, glycine HCl, and citric acid. Examples of alkalizing agents may be but are not limited to sodium hydroxide, potassium hydroxide, ammonium hydroxide, tromethamine, histidine.

As another example, a method (e.g., any of Method 1.0 et seq.) for treating premature ejaculation in a patient in need thereof is herein provided where local anesthetic or SSRI is locally administered to at least one location of a penis of the patient, wherein the location is the frenulum and/or glans of the penis, to thereby treat premature ejaculation in the patient. In certain aspects, the local anesthetic or SSRI antidepressant administration can be to a single location on the penis (e.g. frenulum) or distributed over two or more anatomically distinct portions of the penis (e.g. penile frenulum, penile prepuce/foreskin, glans penis, urethral opening).

Additionally, a method (e.g., any of Method 1.0 et seq.) for prolongation of climax time in a patient in need thereof is provided wherein the method comprises the step of locally administering a local anesthetic, opioid, and/or SSRI to the patient to thereby prolong the climax time in the patient. Administration of local anesthetic, opioid, and/or SSRI can be via transdermal, intramuscular, subcutaneous, subdermal, intradermal, parenteral or implant administration, and can be to a frenulum or prepuce, for example.

A method as disclosed herein (e.g., any of Method 1.0 et seq.) may comprise solutions suitable for administration by injection, which includes aqueous or non-aqueous isotonic sterile solutions, which may contain antioxidants, buffers, bactericides and solutes. The solutions may be presented containing all components in a single unit dose sealed container, for example ampoules or vials. The solutions of, e.g., any of Method 1.0 et seq., may be stored in a freeze dried form (lyophilized) requiring only the addition of the sterile liquid carrier, for example, water, immediately prior to use.

Standard needle injection (e.g., needle injection), as described in any of Methods 1.0 et seq., contemplates a vial of medication, presuspended in solution, or in powder/lyophilzed form, is prepared in a kit with all supplies to prepare and administer a standard needle injection. The steps for using the injection include, e.g.: (1) selecting medication vial and dissolving in diluent if in powder form; (2) wiping the top of this vial with an alcohol swab; (3) Preparing a syringe with a needle to draw up the anesthetic; (4) drawing back the plunger of the syringe; (5) inserting the needle into the vial; (6) injecting the air into the vial to avoid creating a vacuum after withdrawal of the liquid; (7) positioning the tip of the needle in the liquid; (8) drawing back on the syringe plunger while holding the needle in the vial; (9) removing the precise amount of solution; (10) removing the needle/syringe from the vial; (11) changing the larger needle used to draw up the solution for a smaller needle used for injection; (12) evacuating any air in the syringe prior to injecting into the target site; (13) clean skin on penis; (14) inject medication; (15) hold pressure at site for short time e.g. 20 seconds.

Alternatively, a pre-prepared or pre-filled syringe with attached needle and dispensing plunger is sold in sterile fashion and can be used with any of the methods of Method 1.0 et seq, and/or Kit 2.0, et seq. The syringe would be similar to a small lmL insulin syringe with fine, short needle, e.g. 30 gauge. To use, the patient would simply removed guards and covers from syringe, clean skin, insert needle, gently plunge medication into tissue, and then carefully dispose of syringe in safe fashion.

Needle-free jet injection, as described in any of Method 1.0 et seq., contemplates a pre-loaded, low injection pressure, easy-to-use, needle-free jet injection device. Medications injected would be local anesthetic, opioid, and/or SSRI class medication. Instructions would be for application onto the dorsum of the penis anywhere from just proximal to the coronal sulcus of the glans penis to the base of penis possibly after cleaning (e.g. with alcohol swab). In certain aspects the device would be one-time-use only, or else would house a replaceable for a one-time use only cartridge filled with appropriate dose/volume of anesthetic that would permit re-use of the needle free jet injector device with replacement of the medication cartridge.

These particular devices have the benefit of elimination of “needle anxiety” described in background above, and would be easy and safe to use. In the case of short acting agents e.g. anesthetic or SSRI, in one aspect, a one-time use cartridge would permit the medication to be prepared potentially without use of a preservative (e.g. benzyl alcohol). Such preservatives are needed to prevent bacterial overgrowth in multi-use vials. The advantage of not including such preservatives is that they can increase the discomfort of injection. Dosing and application location on the penis can be personalized to reach maximal effect and minimal undesired effects such as penile numbness.

Common needle free jet injection systems, as described in any of Methods 1.0 et seq, include: (a) a chamber for holding an injectable liquid; (b) an orifice for directing pressurized injectable out of the injectable chamber and onto a target region; and (c) a plunger mechanism for ejecting a selectable amount of fluid from within the chamber, through the orifice, and onto a target site spaced at a pre-determined interval from the orifice. For example, needle-free jet injectors which can be used with the present invention can be found in U.S. Pat. Nos. 4,596,556; 4,790,824; 4,940,460; 4,941,880; 4,966,581; 5,064,413; 5,312,335; 5,312,577; 5,383,851; 5,399,163; 5,466,220; 5,503,627; 5,505,697; 5,520,639; 5,649,912; 5,746,714; 5,782,802; 5,893,397; 5,993,412; 6,096,002; 6,132,395; 6,264,629; 6,319,224; 6,383,168; 6,471,669; 6,506,177; 6,572,581; 6,585,685; 6,607,510; 6,641,554; 6,645,170; 6,648,850; 6,676,630; 6,689,093; 6,752,780; 6,752,781; 6,783,509; 6,883,222; 6,935,384; 7,131,961; and 7,156,823, the disclosures of each of which are herein incorporated by reference in their entirety for all purposes. The above list is representative but not meant to be comprehensive or limiting.

The “CAVERJECT™” system as described herein, (e.g., any of Methods 1.0 et seq.), in one aspect refers to a disposable, single-dose, dual chamber syringe system. In one aspect this can include a glass cartridge, which can further contain sterile freeze-dried alprostadil in the front chamber and sterile bacteriostatic water for injection in the rear chamber. In one aspect the system also comprises a local anesthetic (e.g., bupivacaine) Following proper reconstitution instructions, the 10 μg strength syringe can deliver up to 0.5 mL of solution. Each 0.5 mL of solution can contain, approximately, e.g., 10 μg, 324 μg of alpha cyclodextrix, 45 μg of lactose, 23 μg of sodium citrate, and 4.45 mg of benzyl alcohol. The delivery device can be set to deliver a solution volume of, e.g., 0.125, 0.25, 0.375, or 0.5 mL to enable administration of e.g., 2.5, 5, 7.5, or 10 μg of alprostadil.

The age range of patients upon which the methods herein disclosed (e.g., any of Methods 1.0 et seq.) may be utilized may from about 14 years old to about 90 years old, more particularly, from about 14 years old to about 40 years old, and even more particularly, from about 18 years old to about 30 years old. In particular instances, the patient has tried various previous treatments that have not been found to satisfactorily treat the patient's premature ejaculation.

Patients that can be treated by the methods herein disclosed (e.g., any of Methods 1.0 et seq.) may have previously partaken in regimens for treating their premature ejaculation or for prolongation of their climax time. Exemplary regiments can include taking, via oral administration, e.g., a selective serotonin reuptake inhibitor, such as fluoxetine or paroxetine. Other approaches that may have been tried include application of topical anesthetics, such as lidocaine 2% cream, applied to the penis before intercourse. In certain aspects, (e.g., any of Methods 1.0 et seq.) oral administration of a SSRI, and/or application of a topical anesthetic (e.g., lidocaine 2% cream), is combined with the methods herein disclosed (e.g., any of Methods 1.0 et seq.) in order to treat premature ejaculation and/or for prolongation of climax time. In certain aspects, the methods disclosed herein (e.g., any of Methods 1.0 et seq.) may be used in combination with patients undergoing longer duration interventions e.g. botox, neuromodulation, surgical denervation, cryoablation of nerves, or other permanent ways that are used to obliterate conduction along nerves and/or their branches.

In certain aspects the local anesthetic or SSRI is administered on an as-needed basis. Dosing will be determined for, and be particular to, the patient/particular presentation of premature ejaculation, with non-limiting, exemplary amounts provided herein.

Volume of injection and dose of medication would be minimized to achieve adequate, reproducible effects. Volume of solution may be as little as 0.05 mL and as much as 5 mL, but more likely between 0.01 mL and 1 mL.

Compared to topically applied methods (e.g. TEMPE), this home-based injection strategy boasts absence of residue of medication on the penis, rapid time to efficacy, and less likelihood of sensory changes to the sexual partner due to direct transference of medication, and absence of undesirable taste due to residue from creams.

The invention contemplates that the device can be designed and packaged for safe patient self-administration at home or potentially by the physician in the physician office (e.g. for test doses, or for longer acting agents like Botox). The invention contemplates a kit which would permit discreet carrying in personal handbags.

FIG. 1A. The invention contemplates that the injection can be anywhere as described in Method 1.0 seq., and/or described in Kit 2.0 et seq. In one aspect the injection as described in Method 1.0 et seq and/or Kit 2.0 can be to dorsally (e.g., subcutaneously) as shown in FIG. 1A, wherein the injection can be in the range in the area to the left (2) or right (1) of the midline (represented by the black rectangular area). This figure is intended to be exemplary and not limiting in any respect.

FIG. 1B. The invention contemplates that the injection can be anywhere as described in Method 1.0 seq., and/or described in Kit 2.0 et seq. In one aspect the injection as described in Method 1.0 et seq and/or Kit 2.0 et seq., can be around the area of the frenulum (4) and/or to the dorsum (3) area as well. This figure is intended to be exemplary and not limiting in any respect.

FIG. 1C. The invention contemplates that the injection can be anywhere as described in Method 1.0 seq., and/or described in Kit 2.0 et seq. In one aspect the injection as described in Method 1.0 seq., and/or described in Kit 2.0 et seq., can be administered as an infrapubic (6) injection, wherein the injection, in one aspect, is around the abdominal wall (5). This figure is intended to be exemplary and not limiting in any respect. In one aspect, infrapubic pertains to areas of the patient which are located around or below the pubis.

### Example 1

It is contemplated by the present invention, that a male patient with lifelong premature ejaculation and relationship stress proceeds to contact his physician. The physician prescribes a teaching injection of 0.2 mL bupivacaine. The administration of bupivacaine is made dorsally just before the glans, e.g., within 1 cm proximal to the glans. The injection is made subcutaneously to the left or right of midline. The injection anesthetizes the dorsal penile nerve. A 30-32 gauge needle is used, and the need is 10 mm or less (e.g., between 4-10 mm, e.g., 4 mm). The patient self-injects at home and his intravaginal latency time increases more than two-fold with a better side-effect profile compared to previous therapies. In one aspect the patient sees an improvement in prolonging climax from 45 seconds (prior to treatment) to 4 minutes (following treatment).

### Example 2

It is contemplated by the present invention, that a male patient with lifelong premature ejaculation and relationship stress proceeds to contact his physician. The physician prescribes a teaching injection of 0.4 mL bupivacaine. The administration of bupivacaine is made dorsally just before the glans, e.g., within 1 cm proximal to the glans. The injection is made subcutaneously to the left or right of midline. The injection anesthetizes the dorsal penile nerve. A 30-32 gauge needle is used, and the need is 7 mm or less (between 4-7 mm). The patient self-injects at home and his intravaginal latency time increases more than two-fold with a better side-effect profile compared to previous therapies. In one aspect the patient sees an improvement in prolonging climax from 45 seconds (prior to treatment) to 4 minutes (following treatment).

### Example 3

It is contemplated by the present invention, that the male patient described in Example 2 wishes an even greater numbing effect in subsequent injections. In this case, in order to obtain a greater numbing effect, the patient injects half of the teaching injection, 0.2 ml bupivacaine, dorsally just before the glans, e.g., within 1 cm proximal to the glans. The injection is made subcutaneously to the left or right of midline. The injection anesthetizes the dorsal penile nerve. A 30-32 gauge needle is used, and the need is 7mm or less (e.g., between 4-7 mm). The patient injects the other half of the injection, 0.2 ml bupivacaine, into the frenulum subcutaneous space.

### Example 4

A male patient with premature ejaculation is 18 years or old and in a monogamous heterosexual relationship for 6 months. A screening/baseline run-in phase will request at least 4 measurements of IELT over a 4-6 week period. For each sexual episode, partner will record PEP, IELT, date and time of sexual encounter; patient will fill out PEP. To qualify for the treatment phase, patients must have a measured IELT of 2 minutes or less in 75% of valuable events.

A volume of distribution of 0.4 and 0.8 mL will bathe the dorsal penile nerves as they are located just to the right and left of midline. Diffusion should occur across the midline given that the subcutaneous compartment has a low resistance to fluid migration. Diffusion of the drug solution should occur through Buck's fascia to the dorsal penile nerves.

A concentration of bupivacaine (0.25%) is used for local nerve blocks. Doses of administration are of 1 mg of bupivacaine for the 0.4 mL volume and 2 mg for the 0.8 mL group. This is less than 1.5% of the maximum permitted dose. Adverse dose-related effects are not anticipated.

Patients will be randomized to one of 4 treatment groups. Patient and physician will be blinded to assigned treatment. During the treatment phase, the patient will have 4 office visits with an injection drawn up by the research nurse and injected by the physician at each visit. These 4 visits will occur over a minimum of 4 weeks.

A baseline will be taken over a period of 6 weeks, during which patient and partner record at least 4 sexual encounters where IELT and PEP scores are collected. These are brought to visit #3 to determine eligibility for treatment phase.

Subsequently, patient will be evaluated for eligibility for “treatment phase”—must have either: (1) IELT of 2 minutes or less in at least 75% or sexual episodes; or (2) IELT of 3 minutes or less in at least 75% of sexual episodes with PEP score indicating accompanying psychosocial or relationship distress.

Patient will receive their clinician-administered injections and then be observed for 15 minutes for any immediate local or systemic adverse events. Vital signs (blood pressure, heart rate, respiratory rate) and examination of the injection site will be performed prior to discharge home to ensure absence of any adverse reactions. Patient will go home and have sexual relations with the same partner as in baseline evaluation.

Home data collection for every sexual episode will include:

Recordation of: (1) time of sex and IELT in log book; (2) Fill out PEP; and

Patient will fill out (1) adverse events form including visual analog pain score; (2) PEP.

A final debriefing will include collection of any outstanding scoresheets. In one aspect the patient in the treatment groups will report an improvement in prolonging climax from 45 seconds (prior to treatment) to 4 minutes (following treatment).

1. Method of treating premature ejaculation in a patient in need thereof, wherein the method comprises administering an effective amount of a solution comprising a local anesthetic and/or SSRI and/or opioid, wherein the solution is injected directly into the penis of the patient, e.g., by needle injection, needle-free injection, and/or microneedle injection.

2. Method of claim 1, wherein the SSRI or local anesthetic is administered between about 1 minute and 12 hours prior to engaging in sexual intercourse.

3. Method of claim 1, wherein the patient self-administers the solution comprising the anesthetic or SSRI or opioid prior to engaging in sexual activity.

4. Method of claim 1, wherein the local anesthetic is selected from the group consisting of: Lidocaine, marcaine, tetracaine, bupivacaine, mepivacaine, Articaine, Carticaine, Cinchocaine/Dibucaine, chlorprocaine, dibucaine, etidocaine, hexylcaine, procaine, ketamine, pramoxine, dyclonine and phenol, Levobupivacaine, Piperocaine, Ropivacaine, Trimecaine, Benzocaine, Chloroprocaine, Cocaine, Cyclomethycaine, Dimethocaine/Larocaine, carbisocaine, ciprocaine, butanilicaine and trimecaine, Propoxycaine, Procaine, Prilocaine, Proparacaine, Tetracaine/Amethocaine, Saxitoxin and Tetrodotoxina.

5. Method according to claim 1, wherein the SSRI is selected from the group consisting of: Paroxetine, dapoxetine, Clomipramine, Fluoxetine, sertraline, flibanserine.

6. Method according to claim 1 wherein the injection further comprises opioids e.g. Tramodol.

7. Method according to claim 1 wherein the injection is administered using a standard needle injection directly to the penis.

8. The method of claim 7, wherein the gauge of the needle is greater than 23.

9. The method of claim 7 wherein the length of the needle is between 3-10 mm.

10. Method according to claim 1, wherein the injection is administered using a pre-filled disposable single use syringe, (e.g., SIMPLIST™ from Becton Dickenson)

11. Method according to claim 1, wherein the injection is administered using an auto-injection pen (e.g., Apidra SOLOSTAR™, Lantus SOLOSTAR™, BYETTA™, SYMLINPEN 60™, HumaPen LUXURA HD™, Humalog KWIKPEN™, Humulin Pen, VICTOZA™, NOVOLOG MIX FLEXPEN™, NOVOPEN JUNIOR™, NOVO PEN 3™, LEVEMIR FLEXPEN™, AUTOPEN™, FOLLISTIM PEN™ and CAVERJECT IMPULSE™).

12. Method according to claim 1, wherein the injection is administered via a microneedle injection, e.g., a microneedle dermal patch, e.g., 3M HOLLOW MICRONEEDLE SYSTEM™ or 3M SOLID MICRONEEDLE SYSTEM.

13. Method according to claim 1, wherein the injection is administered using an insulin pen (e.g., 29-32 gauge; e.g., about 4-6 mm)

14. Method according to claim 1, wherein the injection is administered via a needle-free jet injection e.g. Bioject or Pharmajet Product.

15. Method according to claim 1, wherein the volume of the injected solution is between about 0.05 ml-2.0 ml, e.g., about 0.05 ml, 0.1 ml, 0.2 ml, 0.3 ml, 0.4 ml, 0.5 ml, 1.0 ml, 1.5 ml, or 2.0 ml.

16. Method according to claim 1, wherein the injection is administered via transdermal, intramuscular, subcutaneous, subdermal, intradermal or implant administration.

17. Method according to claim 1, wherein the injection is a subcutaneous injection.

18. A kit comprising an injection system (e.g., standard needle injection, microneedle injection, or needle-free jet injection) of claim 1, wherein the injection system comprises, for example, (1) a pre-filled syringe with a 29-34 gauge needle 4-10 mm long; (2) medication in liquid form; (3) alcohol swab to clean skin prior to drawing up medication; (4) instructions for use with diagrams, and wherein the kit offers the benefit of allowing a patient to administer the medication himself or herself.

19. Method of claim 1, wherein a therapeutically effective amount of a local anesthetic and/or SSRI is administered as a test dose prior to a long lasting intervention e.g. Botox, cryoablation nerves, neuromodulation of nerves, surgical denervation, radiofrequency ablation of nerves

20. Method of claim 1, wherein the injection is administered subcutaneously to the dorsum of the penis anesthetizing the dorsal penile nerves within 10 mm to the left and/or right of the dorsal midline of penis, either proximally or distally, or the branches of the dorsal penile nerves just proximal to the glans penis along the midline or broader dorsal surface of the penis.

21. Method of claim 1, wherein the injection is administered subcutaneously to the sides and/or ventrum of the penis, and/or frenulum.
